(19) United States
(12) Patent Application Publication
(10) Pub. No.: US 2019/0255175 A1
(43) Pub. Date: Aug. 22, 2019

(54) ACTIVATION OF TRPA1+ NOCICEPTORS IN THE VAGUS NERVE ATTENUATES SYSTEMIC INFLAMMATION

(71) Applicant: THE FEINSTEIN INSTITUTE FOR MEDICAL RESEARCH, Manhasset, NY (US)

(72) Inventors: Kevin J. Tracey, Old Greenwich, CT (US); Sangeeta S. Chavan, Syosset, NY (US); Harold A. Silverman, Plainview, NY (US)

(73) Assignee: THE FEINSTEIN INSTITUTE FOR MEDICAL RESEARCH, Manhasset, NY (US)

(21) Appl. No.: 16/274,370

(22) Filed: Feb. 13, 2019

Related U.S. Application Data
(60) Provisional application No. 62/631,585, filed on Feb. 16, 2018.

(51) Int. Cl. A61K 41/00; A61K 31/4439; A61P 29/00; A61P 31/04

(52) U.S. Cl. CPC A61K 41/0057; A61P 31/04; A61P 29/00; A61K 31/4439

(57) ABSTRACT

Methods are disclosed for treating inflammation and sepsis and for promoting thermoregulation by activating neurons expressing TRPA1 in the vagus nerve.

### CROSS-REFERENCE TO RELATED APPLICATIONS

This application claims the benefit of U.S. Provisional Patent Application No. 62/631,585 filed on Feb. 16, 2018, the contents of which are incorporated herein in their entirety.

### STATEMENT OF GOVERNMENT SUPPORT

This invention was made with government support under grant number GM118182 awarded by the National Institutes of Health. The government has certain rights in the invention.

### BACKGROUND OF THE INVENTION

Throughout this application various publications are referred to as superscripts. Full citations for these references may be found at the end of the specification immediately preceding the claims. The disclosures of these publications are hereby incorporated by reference in their entireties into the subject application to more fully describe the art to which the subject application pertains.

Afferent vagus nerve signaling is a critical component of maintaining an organism's homeostasis. The afferent signaling of the vagus nerve helps maintain physiological states dealing with digestion, cardiac function, respiratory function and immunity¹⁻³. The afferent vagus nerve carries the signal for interleukin 1β (IL-1β)-induced febrile response⁴, as well as the signaling for sickness behavior in rodents; including lethargy and anorexia when challenged with inflammatory stimuli⁵⁻⁷. Advancements in neuroscience and immunology along with recent technological advancements have begun to show that inflammatory molecules such as lipopolysaccharide (LPS) and pro-inflammatory cytokines such as tumor necrosis factor (TNF) and IL-1β can cause increases in vagus nerve signaling differentially⁸⁻¹¹.

The efferent arm of the inflammatory reflex has been increasingly well studied. It has been shown that the afferent IL-1β signals continue to the efferent splenic nerve⁹. This has been identified through electrical recordings of the rat splenic nerve, in which IL-1β induced signaling is ablated by a subdiaphragmatic vagotomy⁹. Similarly, previous work has identified that not just the splenic nerve, but the spleen is essential to the inflammatory reflex¹²⁻¹⁴. The efferent reflex signaling has been shown to travel through the splenic nerve, with these fibers forming a synapse with ChAT+ T-cells, which can release acetylcholine that binds to the α7 subunit of the nicotinic acetylcholine receptor (nAChR) on cytokine-producing macrophages in the spleen inhibiting TNF production¹³⁻¹⁵.

IL-1β's link to the febrile response through the vagus nerve has been well documented⁴, as has the paradoxical thermoregulatory response that occurs within a mouse based on their environmental temperatures. When housed at thermoneutral temperature (˜30° C.), IL-1β induced a fever or hyperemia response; however, when housed below thermoneutral temperature, the opposite response occurs, and the mouse becomes hyperthermia¹⁶.

The present application addresses the need for improved methods for treating inflammation and sepsis, and promoting thermoregulation.

### SUMMARY OF THE INVENTION

Method are provided for suppressing or ameliorating inflammation, promoting thermoregulation, and/or treating sepsis in a subject comprising activating neurons expressing the nociceptor ion channel designated “transient receptor potential cation channel, subfamily A, member 1” (a/k/a “transient receptor potential ankyrin 1” or “TRPA1”) in the subject's vagus nerve effective to suppress or ameliorate inflammation, promote thermoregulation, and/or treat sepsis in a subject.

Methods are also provided for suppressing or ameliorating fever in a subject and for activating an immune response in an immunosuppressed subject comprising administering to the subject an antagonist of TRPA1 in amount effective to suppress or ameliorate fever in a subject or activate an immune response in an immunosuppressed subject.

### BRIEF DESCRIPTION OF THE DRAWINGS

FIG. 1A-1C. Stimulation of sensory TRPA1+ vagus fibers attenuates serum TNF levels in endotoxemic mice. A) Wild type mice were treated with optovin directly to the vagus nerve, followed by exposure to no light (n=9) or 405 nm light (n=10). Mice were also subjected to a proximal vagotomy (n=12) cutting off afferent signaling to the brain. These mice were also treated with optovin and stimulated with 405 nm light. B) TRPA1 KO mice were treated with optovin directly to the vagus nerve. The mice were then treated with no light (n=9) or 405 nm light (n=8). C) α7 KO mice were treated with optovin directly to the vagus nerve. The mice were then treated with no light (n=10) or 405 nm light (n=12). Two asterisks indicate p<0.01.

FIG. 2A-2D. Pharmacological Stimulation of TRPA1+ neurons attenuates TNF levels in serum and spleen in endotoxemic mice. A) Serum: wildtype mice received intraperitoneal administration of polygodial (5 mg/kg) (n=6) or vehicle (n=5). After 30 minutes, mice were subjected to endotoxemia (0.1 mg/kg endotoxin, i.p.). Blood was collected after 90 min and serum TNF levels were determined by ELISA. *p<0.05. B) Spleen: wildtype mice received intraperitoneal administration of polygodial (5 mg/kg) (n=7) or vehicle (n=6). After 30 minutes, mice were subjected to endotoxemia (0.1 mg/kg endotoxin, i.p.). Spleens were collected after 90 min and serum TNF levels were determined by ELISA. *p<0.05. C, D) Pharmacological Stimulation of TRPA1+ neurons fail to attenuate TNF levels in endotoxemic TRPA1 KO mice. C) Serum: TRPA1 KO mice received intraperitoneal administration of polygodial (5 mg/kg) (n=9) or vehicle (n=8). After 30 minutes, mice were subjected to endotoxemia (0.1 mg/kg endotoxin, i.p.). Blood was collected after 90 min and serum TNF levels were determined by ELISA. D) Spleen: TRPA1 KO mice received intraperitoneal administration of polygodial (5 mg/kg) (n=9) or vehicle (n=8). After 30 minutes, mice were subjected to endotoxemia (0.1 mg/kg endotoxin, i.p.). Spleens were collected after 90 min and serum TNF levels were determined by ELISA.

FIG. 3. TRPA1 is required for IL-1β induced vagus nerve signaling. Vagus nerve firing rate in 60s-Bins (mean±SEM) starting 5 min prior to IL-1β injection (350 ng/kg) indicated by arrow followed by 5 min post injection time.

FIG. 4A-4C. TRPA1 KO mice exhibit spontaneous inflammatory phenotype as indicated by increased levels of IL-6 (A), IL-1β (B) and KC/GRO (C). Wild type and TRPA1 KO mice were injected with saline and euthanized 3 hours later. *p<0.05, **p<0.01, ***p<0.005.

FIG. 5A-5B. A) IL-1β does not induce hypothermic response in TRPA1 KO mice. Body temperature was recorded using an ETA-F10 temperature implant (DSI New Brighton, Minn.) that was placed in the peritoneal cavity and fixed to the peritoneal wall. After recording the baseline temperature for 1 hr, rmIL-1β (5.0 μg/kg) was administered by i.p injection. Body temperature of wildtype (n=4) and TRPA1 KO mice (n=4) was recorded for 5 hours post injection. ****p<0.0001. B) IL-1β induces a dose-dependent hypothermic response in mice. After recording the baseline temperature for 1 hr, rmIL-1β (0 [n=4], 0.5 [n=4], 50.0 [n=3] μg/kg) was administered by i.p injection. Body temperature was recorded for 5 hours post injection.

FIG. 6A-6F. TRPA1 KO exhibit increased sepsis mortality, while activation of TRPA1 enhances sepsis survival. A) TRPA1 KO (dotted line) mice have increased morality rate in sepsis in comparison to WT mice (solid line) (p<0.05). B) TRPA1 KO mice have a higher percentage of weight loss after CLP (two-way ANOVA, p<0.001). Disease severity scores C) MSS and D) M-CASS were significantly higher in TRPA1 KO mice in comparison to WT three days post CLP. Disease severity scores E) MSS and F) M-CASS were significantly higher in TRPA1 KO mice in comparison to WT six days post CLP.

### DETAILED DESCRIPTION OF THE INVENTION

The invention provides a method of suppressing or ameliorating inflammation, promoting thermoregulation, and/or treating sepsis in a subject comprising activating neurons expressing the nociceptor ion channel designated “transient receptor potential cation channel, subfamily A, member 1” (a/k/a “transient receptor potential ankyrin 1” or “TRPA1”) in the subject's vagus nerve effective to suppress or ameliorate inflammation, promote thermoregulation, and/or treat sepsis in a subject.

In one embodiment, the method of suppressing inflammation, promoting thermoregulation, and/or treating sepsis is activated by selectively stimulating vagus nerve fibers expressing TRPA1 nociceptors in the subject. Preferably, the method comprises activating vagus nerve afferents.

In one embodiment, the TRPA1-positive neurons are activated by administering a TRPA1 nociceptor agonist to the subject. In one embodiment, a photochemical activator is administered to the subject and TRPA1+ nociceptors are activated using optogenetic stimulation. For example, optovin can be administered to the subject, or the vagus nerve of the subject, and TRPA1+ nociceptors activated using optogenetic stimulation.

In different embodiments, the subject can have one or more of sepsis, endotoxemia, septicemia or septic shock.

Preferably, activation of TRPA1+ neurons attenuates serum levels of one or more pro-inflammatory cytokines, such as for example, tumor necrosis factor (TNF), interleukin-6 (IL-6), interleukin-1β (IL-1β), and/or KC/GRO(CXCL1).

The invention also provides a method of suppressing or ameliorating fever in a subject comprising administering to the subject an antagonist of TRPA1 in amount effective to suppress or ameliorate fever in a subject.

The invention further provides a method of activating an immune response in an immunosuppressed subject comprising administering to the subject an antagonist of TRPA1 in amount effective to induce an immune response in a subject.

The subject can be any mammal and is preferably a human.

As used herein, to “treat” a disease or condition means to ameliorate a sign or symptom of the disease or condition.

Agonists of TRPA1 are known in the art and include, but are not limited to, optovin, polygodial, resveratrol, ASP 7663 (Tocris), JT010 (Tocris), 4-Oxo-2-nonenal, allicin, allyl isothiocyanate, cannabidiol, gingerol, icilin, hepoxilins A3 and B3, 12S-Hydroperoxy-5Z,8Z,10E,14Z-eicosatetraenoic acid, 4,5-Epoxyeicosatrienoic acid, and supercinnamaldehyde. Antagonists of TRPA1 are also known in the art and include, but are not limited to, AM0902 A967079, AP18, HC030031 and TCS5861528 (all from Tocris), ALGX-2513, ALGX-2541, ALGX-2563, ALGX-2561 and ALGX-2542.

“And/or” as used herein, for example, with option A and/or option B, encompasses the embodiments of (i) option A, (ii) option B, and (iii) option A plus option B.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Materials and Methods

Animals:

All experimental protocols were approved by the Institutional Animal Care and Use Committee (IACUC) at the Feinstein Institute for Medical Research, Northwell Health, which follows the NIH guidelines for ethical treatment of animals. Male B6.129F and TRPA1 KO mice were purchased from Jackson Labs and used between 8 and 20 weeks of age. TRPV1-Cre and TRPV1-DTA mice were bred at the Feinstein Institute for Medical Research and used between the same age range. Mice were housed under reverse day/light cycle and had access to food and water ad libitum. Food was withheld for the 3-4 hours prior to nerve recording; animals continued to have access to water

Surgical Isolation of Cervical Vagus Nerve:

Mice were induced with general anesthesia using isoflurane at 2.5% in 100% oxygen at a flow rate of 1 L/min. Once anesthetized, the mouse was moved to a supine position with the isoflurane maintained at 2.0% during surgery. The nerve was then isolated as described in previous publications^{10,11,17}. The core body temperature was monitored with a rectal probe and maintained around 37° C. with a heating pad and heat lamp. Prior to recording and nerve placement within the cuff, the electrode was submerged briefly in saline solution.

Recording Procedure:

The electrophysiological signals were digitized from the vagus nerve using a Plexon data acquisition system (Omniplex, Plexon Inc., Dallas, Tex.). Recordings were sampled at 40 kHz with a 120 Hz filter and 50 gain. All signals were recorded from a bipolar cuff electrode referenced to the animal ground placed between the right salivary gland and the skin. In experiments with IL-1β challenge, following acquisition of the baseline activity (10 min), 350 ng/kg recombinant human IL-1β (eBioscience, San Diego, Calif.) was administered intraperitoneally; recordings were then continued for 10 min post-injection.

Recording Analysis:

Analysis of raw recordings was done on Spike2 software (version 7, CED). Raw recordings were filtered (using a high pass filter) and smoothed. To identify neural signals, a user-specific adaptive threshold was employed. All signals were identified as neuronal or non-neuronal. Non-neuronal signals, which consisted mainly of cardiac and respiratory components, were manually removed. All recordings with no identifiable neuronal spikes were removed from analyses.

Optical Stimulation Procedure:

Optical stimulation was generated by Thor Labs LED driver DC4100, with a 405 nm LED model M405L3 (Newton, N.J.). Sham mice underwent surgical control up to the point of identifying the cervical vagus nerve, without isolating it from the bundle. In both conditions 2 μl of optovin was applied directly on the nerve for 2 minutes prior to stimulation. Sham animals received no light applied to the nerve. The stimulated group received 405 nm light 1000 mA, 10 hz, with a 10% duty cycle for 5 minutes with an approximate power of 80-85 μW. Animals then recovered for 2 hours and were administered LPS (8 mg/kg) via intraperitoneal (ip) injection. Mice were euthanized 1.5 hours after LPS administration, with serum collected.

Vagotomy Procedure:

All vagotomized animas had their vagus nerve isolated. Prior to the vagotomy a 6.0 suture was tied around the vagus nerve. The nerve was then cut proximally or above (blocking left vagus nerve signaling to the brain while signaling to the periphery remained intact) to where 2 μl of optovin was to be added. All mice were stimulated using the same parameters as previously indicated. Animals then recovered for 2 hours and were administered LPS (8 mg/kg) via intraperitoneal (ip) injection. Mice were euthanized 1.5 hours after LPS administration, with serum collected.

IL-1β Induced Inflammation:

Mice were injected with 3.2 μg/kg IL-1β ip or saline. Three hours later the mice were euthanized, and blood, spleen, and two brain regions collected. Blood was collected by cardiac puncture. To obtain serum from these samples, blood was allowed to clot for approximately 45 min to 1 h. The blood was then spun at 5,000 rpm for 10 min and 10,000 For 2 min. Supernatants (serum) were collected and stored at −20° C. prior to use. The spleen was homogenized using a bullet blender homogenizer (Next Advance, Averill Park, N.Y., USA) and the recommended bead homogenization kit and protocol in a 4° C. walk-in refrigerator. The homogenized sample was then spun down at 15,000 rpm for 10 min with the supernatants collected and stored at −20° C. Splenic protein level was measured using the Bradford assay. IL-6, IL-1β, KC/Gro (CXCL1), and IL-1β for both serum and spleen were measured on a custom mouse inflammatory electrochemiluminescent kit (Meso Scale Discovery, Gaithersburg, Md., USA) according to manufacturer's recommendations. The brain stem region was collected on ice, using a binocular dissection microscope and placed in the same tube. Brain regions were snap frozen on dry ice and transferred to storage at −80° C.

Thermoregulation:

Mice were anesthetized with 2.5% isoflurane with an oxygen flow of 1 L/min. Once anesthetized, the mouse was moved to a supine position with the isoflurane maintained at 2.0% during surgery. A midline incision was made above the peritoneal cavity, with a ETA-F10 temperature implant (DSI New Brighton, Minn.) placed in the peritoneal cavity and tacked to the peritoneal wall. The cavity was then closed, and the mouse monitored every day for 3 days. The mouse was allowed to recover for a minimum of five days prior to any manipulation. The mice were placed on the DIS with baseline ip body temperature recorded for 1 hour prior to IL-1β or saline injection. The ip body temperature recording then continued for 5 hours post injection.

Nodose Ganglion Patech Clamp Recordings:

Whole-cell patch clamp recordings were made with glass pipettes pulled on a P-97 electrode puller (Sutter Instruments, Novato, Calif.). The resistance of the pipettes was 2-4 MΩ when filled with an intracellular solution (in mM: CsCl 140, HEPES 10, EGTA 5, Mg-ATP 2, NaGTP 0.3, MgCl2 2, Phosphocreatine 10, pH 7.25 adjusted with CsOH). Cells were superfused at a rate of 2 ml/min with an external bath solution containing the following (in mM): 150 NaCl, 2.5 KCl, 10 HEPES, 10 D-glucose, 2 CaCl2, and 1 MgCl2, pH 7.3-7.4. Experiments were performed at room temperature (22-24° C.). Currents from neurons were monitored with an Multiclamp 700B amplifier (Molecular Devices, Union City, Calif.), acquired through Digidata 1550B (Molecular Devices) onto a computer using pClamp 11 software (Molecular Devices). Data were analyzed using Clampfit 11 (Molecular Devices). All chemicals used for electrophysiological recordings were purchased from Sigma. All drugs and solutions were made fresh from drug stock solutions.

Statistical Analysis.

Data are presented as individual samples, mean±SEM. Two-way ANOVA, paired t-test, and Mann-Whitney U tests were used to examine statistical significance using PRISM 6 (Graphpad, La Jolla, Calif.).

### Results and Discussion

Treating Inflammation:

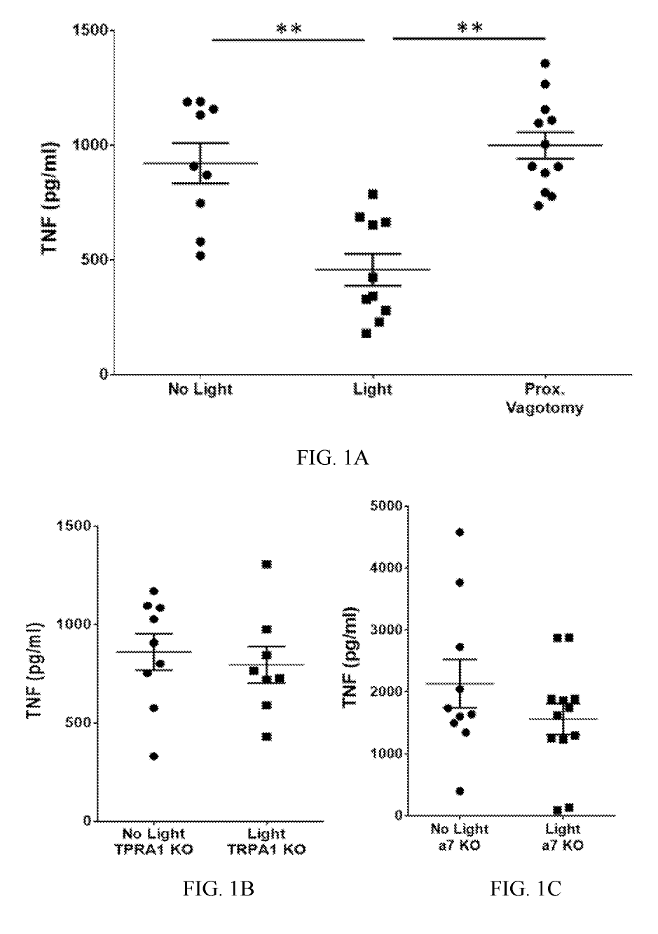

Vagus nerve stimulation has been used as a treatment for peripheral inflammation in both rodents and humans¹⁸⁻²¹. TRPA1 is an ion channel that has been indicated in neural sensing of inflammation within the skin and lungs 22-24. To determine TRPA1's role in the inflammatory reflex, photochemical stimulation of TRPA1+ fibers of the vagus nerve was carried out using a molecule called optovin. Optovin selectively binds to TRPA1 making it photoactive to 405 nm light²⁵. To directly stimulate the TRPA1+ vagus nerve fibers, optovin was applied directly to nerve, with one group unstimulated and one group stimulated with the 405 nm light for 5 minutes with LPS (8 mg/kg) administer 2 hours later. Stimulation of TRPA1+ fibers significantly reduced TNF levels in response to LPS (FIG. 1A). To identify the afferent nature of this signal a vagotomy was performed above the optically stimulated area of the nerve, cutting the signal off from the brain, while keeping the signaling pathway to the periphery intact. The proximally vagotomized animals had no significant reduction in TNF levels (FIG. 1A) identifying the afferent nature of TRPA1's inflammatory signaling though the vagus nerve. Similarly, no significant reductions in TNF levels were observed in TRPA1 knock-out (KO) mice (FIG. 1B). To further investigate TRPA1's role as an afferent mediator of the inflammatory reflex, optovin was applied to the vagus nerve of α7 nAChR KO mice and stimulated as previously indicated followed by an LPS (8 mg/kg) challenge. Optical stimulation of TRPA1+ fibers of the vagus nerve did not significantly reduce serum TNF levels (FIG. 1C), linking TRPA1's signaling to a known mechanism within the inflammatory reflex.

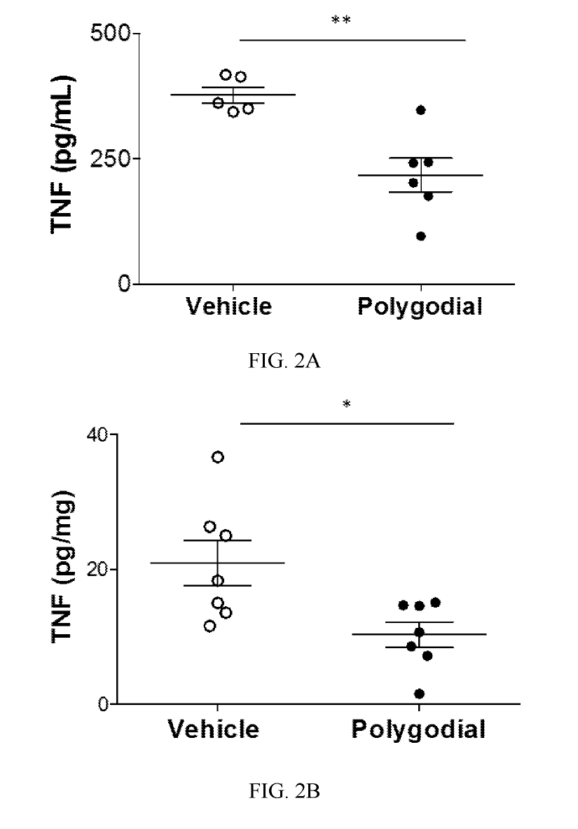

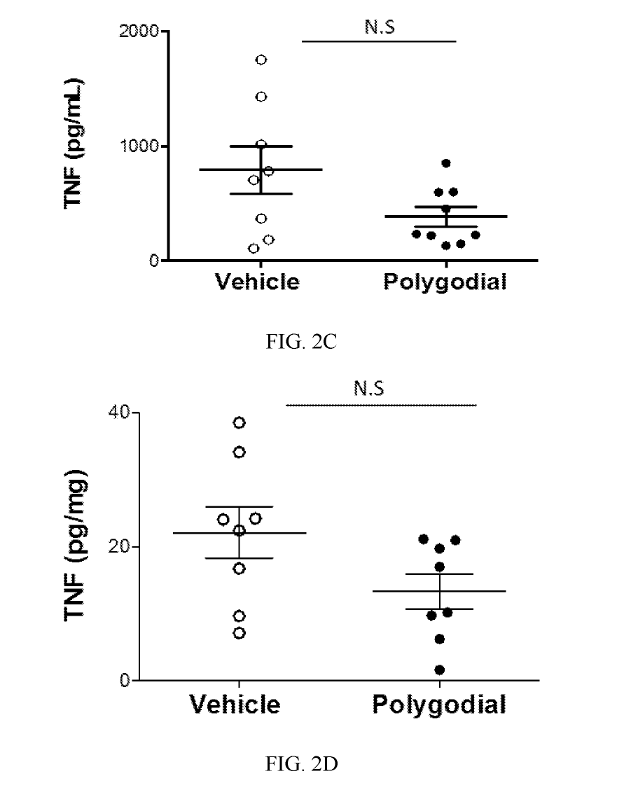

Pharmacological stimulation of TRPA1+ neurons using polygodial also attenuates TNF levels in serum and spleen in endotoxemic mice (FIG. 2A-2B). In contrast, no significant reduction was observed in endotoxemic TRPA1 KO mice (FIG. 2C-2D).

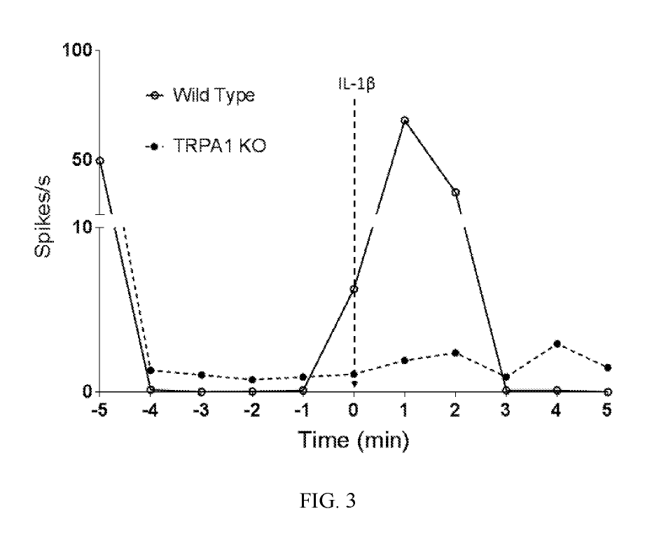

To recognize how knocking out TRPA1 further effects inflammatory signaling, TRPA1 KO and WT mice were injected with either saline or IL-1β (3.2 ng/kg). IL1β induces activation of the vagus nerve in wild type mice; however, IL-1β fails to induce activation of the vagus nerve in TRPA1 KO mice indicating that TRPA1 is necessary for IL-1β induced vagus nerve signaling (FIG. 3).

To further determine fiber specificity, a TRPV1 cell depletion model was employed by breeding TRPV1-Cre mice with ROSA-DTA mice. No significant enhancement of vagus nerve activity was observed due to IL-1β administration, indicating that TRPV1+ fibers of the vagus nerve carried the IL-1β induced signal. To identify if TRPV1 itself is necessary for propagation of the signal, recordings were done in a genetic TRPV1 knockout (TRPV1 KO) mouse. Unlike the cell depleted mice, the TRPV1 KO IL-1β induced a significant increase (Mann-Whitney, p>0.05, U=4) in vagus nerve activity, indicating that genetically ablating the TRPV1 ion channel is not necessary to significantly reduce IL-1β vagus nerve signaling. To determine a molecular target on TRPV1+ neurons mediating afferent IL-1β signaling, the cation channel TRPA1 was identified. TRPA1 is found on a subset of TRPV1+ neurons; approximately 30%. However, TRPA1 co-localized with TRPV1 on 97% of TRPA1+ neurons²⁶.

Using patch clamp recordings, IL-1β responses were recorded in nodose ganglion neurons. Application of IL-1β (20 μg/ml) induced calcium influx as indicated by increase of fluorescence in 6 out of 332 (1.8%) sensory nodose ganglion neurons from VGlut2-GCaMP3 mice. Next, whole-cell patch clamp recordings were carried out on the same neurons that responded to IL-1β in calcium influx analysis. When the membrane potential was held at −65 mV, bath application of IL-1β (20 μg/ml) produced slow inward currents in 4 out of 332 (1.2%) nodose neurons. The amplitude of IL-1β induced currents are 108±23 pA (n=4). Inward currents after IL-1β application were completely blocked by a specific TRPA1 antagonist AM0902, indicating that IL-1β induced currents could be mediated by the TRPA1 channel. In current-clamp mode, IL-1β reduced action potential threshold from −25±2 mV to −31±3 mV (n=4). Together, these data show that IL-1β increases membrane excitability via TRPA1 activation in a subset of nodose sensory neurons. Using Ca-influx assay, it was found that the nodose ganglia consist of neurons that selectively respond to capsaicin (TRPV1-positive), polygodial (TRPA1-positive), and IL-1β. The vast majority of IL-1β-responsive neurons also contained TRPA1, suggesting a potential overlap in their neuronal expression. To further determine the potential interaction of TRPA1 with IL-1β, nodose ganglion neurons from TRPA1 KO mice were examined in a culture system. Neurons responding to IL-1β also responded to TRPA1 activation, and in the absence of TRPA1, IL-1β failed to induce activation of nodose sensory neurons. IL1R1 co-localizes with TRPA1 on nodose ganglion neurons, as was determined using immunohistochemical staining.

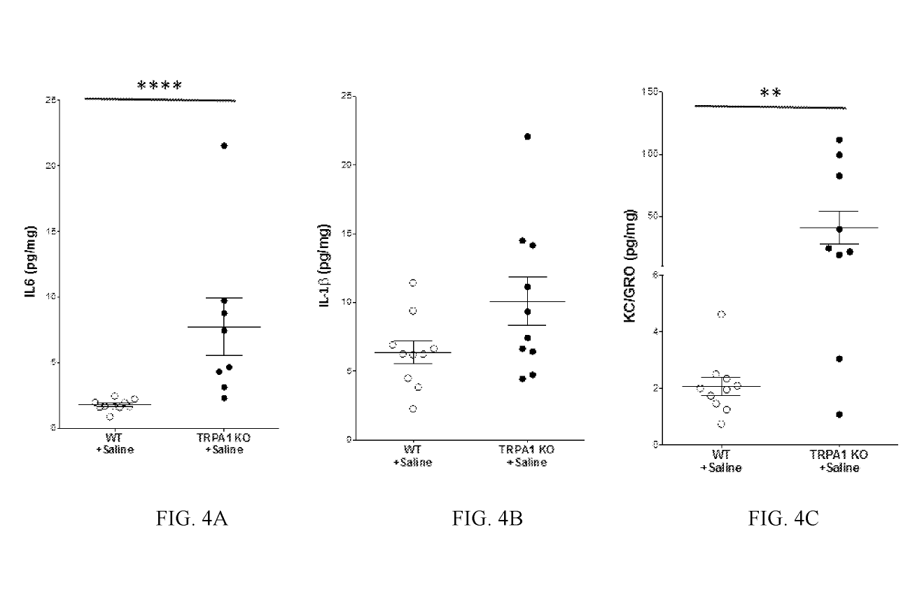

The serum and splenic tissue of these mice were then analyzed for known downstream signaling molecules of IL-1β, IL-6, IL-1β and KC/GRO (CXCL1). Significant increases in both the serum (FIG. 4A-4C) and spleen of TRPA1 KO mice were observed, with further increases observed after IL-1β administration. When taking these results with the previous work, this links TRPA1 as the afferent on-switch of the inflammatory reflex. Exacerbated inflammatory responses in the spleen and serum mediated by both LPS and TNF link directly to the afferent vagus nerve, splenic nerve, spleen, and α7 nAChR through the initiation of TRPA1, indicating that TRPA1 has an essential role in afferent inflammatory signaling and regulation.

Promoting Thermoregulation:

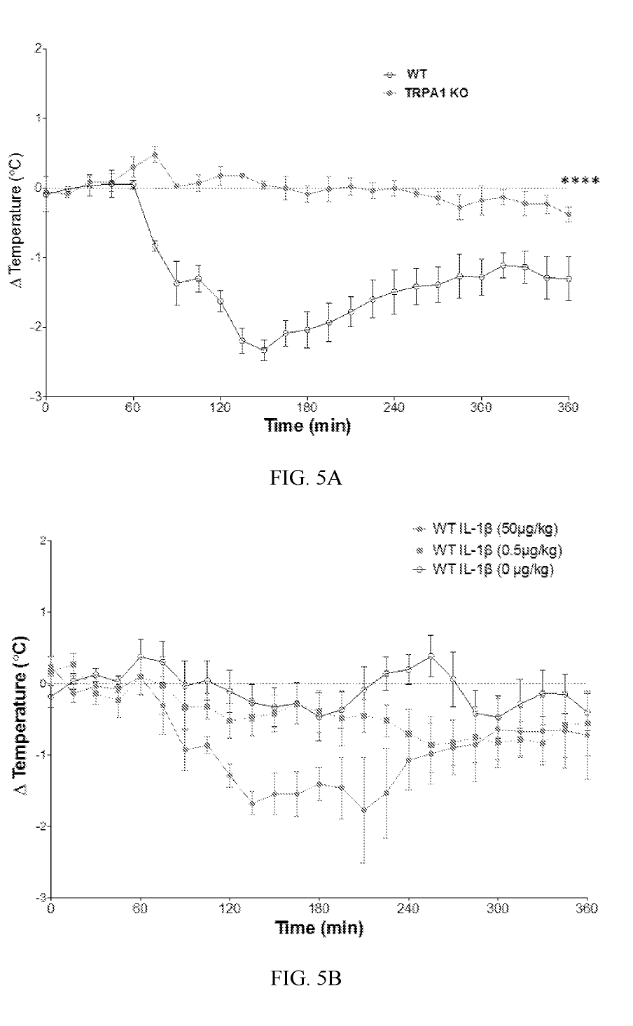

Thermoregulation, specifically in cold sensing, is another major function of the TRPA1 ion channel. When housed at approximately 23° C., WT mice injected with murine IL-1β (5 μg/kg) have significant decrease in body temperature (t-test, p<0.0001) peaking at a temperature change of −2.3±0.15° C., while TRPA1 KO mice have no significant change in body temperature after IL-1β (5 μg/kg) peaking at 0.18±0.40° C. (FIG. 5A). This thermoregulatory response occurs dose dependently with significant decreases in ip body temperature of WT mice in both IL-1β doses of 50 μg/kg (t-test, p<0.0001) and IL-1β 0.5 μg/kg (t-test, p<0.001). However, there was no significant change in ip body temperature after saline (8 μl/mg) (t-test, p>0.05) (FIG. 5B). This indicates a previously unknown mechanism of IL-1β fever singling through TRPA1+ fibers of the vagus nerve, linking TRPA1's role in both thermoregulation as well as afferent inflammatory signaling through the vague nerve.

Treating Sepsis:

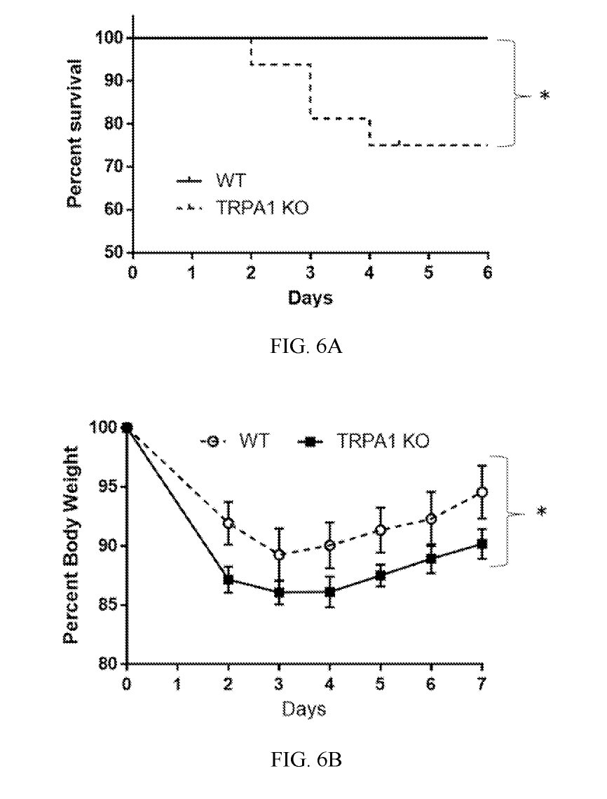

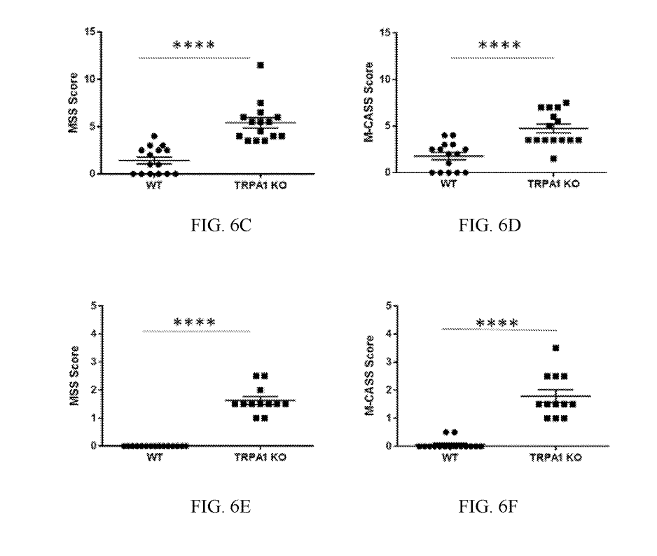

To assess TRPA1's role in disease, cecal ligation and puncture (CLP) was performed to induce sepsis causing a dysregulation in systemic inflammation. In a mild model of CLP, WT mice had 100% survival seven days post-surgery while TRPA1 KO mice had significantly reduced survival rate of 75% (Gehan-Breslow-Wilcoxon test, p<0.05) (FIG. 6A). In accordance with increased disease severity, the TRPA1 KOs exhibited a larger percent loss in body weight compared to WT controls (two-way ANOVA p<0.001). Due to the mild severity of this model, the MSS and M-CASS sepsis disease severity scores were implemented. The murine sepsis score (MSS) is a scoring model for murine sepsis that scores seven clinical variables (appearance, level of consciousness, activity, response to stimulus, eyes, respiration rate, and respiration quality) from 0 to 4 based on appearance. The mouse clinical assessment score for sepsis (M-CASS) is designed to score seven variables (fur aspect, activity, posture, behavior, chest movements, chest sounds, and eye lids) in sepsis survivors. TRPA1 KO mice presented significantly higher severity scores at both day 3 and day 6 (FIG. 6C, 6D, 6E, 6F), demonstrating that TRPA1 is protective in sepsis mortality and disease severity.

The neural-immune interaction described here identifies a newly discovered mechanism of afferent signaling of the inflammatory reflex. The observation that both activation of TRPA1+ fibers of the vagus nerve decreases TNF response in a model of endotoxemia, and that knocking out TRPA1 causes heightened levels of circulating IL-6, IL-1β, and KC/GRO as well as increased sepsis mortality identifies TRPA1 as necessary for proper regulation of inflammatory homeostasis. IL-1β induced inflammation is through the same pathway as indicated by the lack of thermoregulatory response when TRPA1 KO mice are treated with IL-O. The identification of these mechanisms provides new molecular targets that have not been identified for treating systemic inflammatory conditions, as well as IL-1β induced fever and disease.

### REFERENCES

1. Tracey, K. J. The inflammatory reflex. Nature 420, 853-9 (2002).
2. Chavan, S. S., Pavlov, V. A. & Tracey, K. J. Mechanisms and Therapeutic Relevance of Neuro-immune Communication. Immunity 46, 927-942 (2017).
3. Pavlov, V. A., Chavan, S. S. & Tracey, K. J. Molecular and Functional Neuroscience in Immunity. Annu. Rev. Immunol. 36, 783-812 (2018).
4. Watkins, L. R. et al. Blockade of interleukin-1 induced hyperthermia by subdiaphragmatic vagotomy: evidence for vagal mediation of immune-brain communication. Neurosci. Lett. 183, 27-31 (1995).
5. Dantzer, R. Cytokine, sickness behavior, and depression. Immunol. Allergy Clin. North Am. 29, 247-64 (2009).
6. Pavlov, V. A. & Tracey, K. J. The vagus nerve and the inflammatory reflex—linking immunity and metabolism. Nat. Rev. Endocrinol. 8, 743-54 (2012).
7. Goehler, L. E. et al. Vagal immune-to-brain communication: A visceral chemosensory pathway. in Autonomic Neuroscience: Basic and Clinical 85, 49-59 (2000).
8. Niijima, A., Hori, T., Katafuchi, T. & Ichijo, T. The effect of interleukin-1β on the efferent activity of the vagus nerve to the thymus. J. Auton. Nerv. Syst. 54, 137-144 (1995).
9. Niijima, A. The afferent discharges from sensors for interleukin 1β in the hepatoportal system in the anesthetized rat. J. Auton. Nerv. Syst. 61, 287-291 (1996).
10. Steinberg, Benjamin; Silverman, Harold; Robbiati, Sergio; Gunasekaran, Manoj; Tsaava, Téa; Battinelli, Emily; Stiegler, A., Bouton, Chad; Chavan, Sangeeta; Tracey, K.; & Huerta, P. Cytokine-specific neurograms in the sensory vagus nerve. Bioelectron. Med. 3, 7-17 (2016).
11. Silverman, H. A. et al. Standardization of methods to record Vagus nerve activity in mice. Bioelectron. Med. 4, 3 (2018).
12. Huston, J. M. et al. Splenectomy inactivates the cholinergic antiinflammatory pathway during lethal endotoxemia and polymicrobial sepsis. J. Exp. Med. 203, 1623-8 (2006).
13. Rosas-Ballina, M. et al. Splenic nerve is required for cholinergic antiinflammatory pathway control of TNF in endotoxemia. Proc. Natl. Acad. Sci. U.S.A 105, 11008-11013 (2008).
14. Rosas-Ballina, M. et al. Acetylcholine-synthesizing T cells relay neural signals in a vagus nerve circuit. Science 334, 98-101 (2011).
15. Wang, H. et al. Nicotinic acetylcholine receptor alpha7 subunit is an essential regulator of inflammation. Nature 421, 384-8 (2003).
16. Alheim, K. et al. Hyperresponsive febrile reactions to interleukin (IL) 1alpha and IL-1beta, and altered brain cytokine mRNA and serum cytokine levels, in IL-1beta-deficient mice. Proc. Natl. Acad. Sci. U.S.A 94, 2681-6 (1997).
17. Zanos, T. P. et al. Identification of cytokine-specific sensory neural signals by decoding murine vagus nerve activity. Proc. Natl. Acad. Sci. U.S.A 115, E4843-E4852 (2018).
18. Borovikova, L. V et al. Vagus nerve stimulation attenuates the systemic inflammatory response to endotoxin. Nature 405, 458-62 (2000).
19. Bonaz, B. et al. Chronic vagus nerve stimulation in Crohn's disease: a 6-month follow-up pilot study. Neurogastroenterol. Motif. (2016). doi:10.1111/nmo.12792
20. Levine, Y. A. et al. Neurostimulation of the Cholinergic Anti-Inflammatory Pathway Ameliorates Disease in Rat Collagen-Induced Arthritis. PLoS One 9, e104530 (2014).
21. Koopman, F. A. et al. Vagus nerve stimulation inhibits cytokine production and attenuates disease severity in rheumatoid arthritis. Proc. Natl. Acad. Sci. U.S.A 113, 8284-9 (2016).
22. Nassenstein, C. et al. Expression and function of the ion channel TRPA1 in vagal afferent nerves innervating mouse lungs. J. Physiol. 586, 1595-1604 (2008).
23. Bautista, D. M., Pellegrino, M. & Tsunozaki, M. TRPA1: A Gatekeeper for Inflammation. Annu. Rev. Physiol. (2013). doi:10.1146/annurev-physiol-030212-183811
24. Bautista, D. M. et al. TRPA1 Mediates the Inflammatory Actions of Environmental Irritants and Proalgesic Agents. Cell (2006). doi:10.1016/j.cell.2006.02.023
25. Kokel, D. et al. Photochemical activation of TRPA1 channels in neurons and animals. Nat. Chem. Biol. 9, 257-263 (2013).
26. Lopez-Requena, A. et al. Roles of Neuronal TRP Channels in Neuroimmune Interactions. Neurobiology of TRP Channels (CRC Press/Taylor & Francis, 2017). doi:10.4324/9781315152837-15

What is claimed is:

1. A method of suppressing or ameliorating inflammation, promoting thermoregulation, and/or treating sepsis in a subject comprising activating neurons expressing the ion channel designated transient receptor potential cation channel, subfamily A, member 1 (TRPA1) in the subject's vagus nerve effective to suppress or ameliorate inflammation, promote thermoregulation, and/or treat sepsis in a subject.

2. The method of claim 1, wherein TRPA1-positive neurons are activated by administering a TRPA1 nociceptor agonist to the subject.

3. The method of claim 1, wherein the method of suppressing inflammation, promoting thermoregulation, and/or treating sepsis is activated by selectively stimulating vagus nerve fibers expressing TRPA1 nociceptors in the subject.

4. The method of claim 3, comprising activating vagus nerve afferents.

5. The method of claim 1, wherein a photochemical activator is administered to the subject and TRPA1+ nociceptors are activated using optogenetic stimulation.

6. The method of claim 5, wherein optovin is administered to the subject and TRPA1+ nociceptors are activated using optogenetic stimulation.

7. The method of claim 1, wherein the subject has sepsis.

8. The method of claim 1, wherein the subject has endotoxemia, septicemia or septic shock.

9. The method of claim 1, wherein activation of TRPA1+ neurons attenuates serum levels of one or more pro-inflammatory cytokines.

10. The method of claim 9, wherein activation of TRPA1+ neurons attenuates serum levels of tumor necrosis factor (TNF).

11. A method of suppressing or ameliorating fever in a subject comprising administering to the subject an antagonist of TRPA1 in amount effective to suppress or ameliorate fever in a subject.

12. A method of activating an immune response in an immunosuppressed subject comprising administering to the subject an antagonist of TRPA1 in amount effective to induce an immune response in a subject.

13. The method of claim 1, wherein the subject is human.
